(19) United States
(12) Patent Application Publication
(10) Pub. No.: US 2008/0090902 A1
(43) Pub. Date: Apr. 17, 2008

(54) PHENYLALKYLAMINO CARBAMATE COMPOSITIONS

(76) Inventors: Ramendra N. Pandey, Bridgewater, NJ (US); Tracey Mascaro, Hatfield, PA (US); Aniruddha M. Railkar, Ambler, PA (US); James McCool, Sellersville, PA (US); Hinton Clark, Somerset, NJ (US); Stanley Altan, North Brunswick, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/868,555

(22) Filed: Oct. 8, 2007

Related U.S. Application Data
(60) Provisional application No. 60/829,342, filed on Oct. 13, 2006.

(51) Int. Cl. A61K 31/27; A61P 25/00; A61P 25/04; A61P 25/08; A61P 25/14; A61P 25/30

(52) U.S. Cl. 514/489

(57) ABSTRACT

The present invention relates to a composition of a phenylalkylamino carbamate compound that results in improved stability, wherein the composition comprises a phenylalkylamino carbamate compound in a mixture with an effective amount of one or more excipients wherein at least one excipient is dibasic calcium phosphate dihydrate.

This application claims the benefit under 35 U.S.C. 119(e) of U.S. Provisional application Ser. No. 60/829,342 filed Oct. 13, 2006. The complete disclosure of the aforementioned related U.S. Provisional application is hereby incorporated by reference for all purposes.

### FIELD OF THE INVENTION

The invention is directed to a composition of a phenylalkylamino carbamate compound that results in improved stability. More particularly, the compositions comprise a phenylalkylamino carbamate compound in a mixture with dibasic calcium phosphate dihydrate that result in improved stability of the phenylalkylamino carbamate compound.

### BACKGROUND OF THE INVENTION

Phenylalkylamino carbamates are aromatic compounds with a primary aliphatic amine and a carbamate group and are described in U.S. Pat. Nos. 5,705,640, 5,756,817 and 6,140,532, which are incorporated herein by reference. These compounds are pharmaceutically useful for treating CNS disorders, such as pain, depression, anxiety, epilepsy, stroke, dementia and Parkinson's disease. They are soluble and membrane permeable. However, they are susceptible to degradation above pH 5.0, which limits the shelf life of the compounds and compositions thereof. Therefore, there is a need to develop a robust composition of a phenylalkylamino carbamate compound with improved stability of the compound. It is an object of the present invention to provide such a robust composition.

It has previously been disclosed that large particle sizes of dibasic calcium phosphate dihydrate (DCPD) when formulated as a tablet with aspirin has reduced the propensity of aspirin to degrade to salicylic acid and acetic acid compared to smaller particle sized DCPD (Landin et al., 1994, Int. J. Pharm. 107:247-249; Landin et al., 1995, Int. J. Pharm. 123:143-144). The mechanism for the degradation of aspirin to salicylic acid and acetic acid is hydrolysis (Leesen and Mattocks (1958) J. Am. Pharm. Sci. Ed., 67:329-333). The poorer stability of tablets containing powdered material of DCPD as compared to aggregated material was attributed to a greater propensity of smaller particle size DCPD to lose more water (Landin et al., 1994, 1995, supra).

U.S. Pat. No. 6,462,022 discloses the use of large particle sized DCPD (described as having a specific surface area of less than 1.5 m²g⁻¹ prior to compaction or tabletting) in a lisinopril formulation/composition to reduce the amount of the lisinopril degradation product DKP (diketopiperazine) that is formed, thereby increasing the shelf-life of tablets formulated with the larger sized DCPD, particularly those with low doses of lisinopril.

### SUMMARY OF THE INVENTION

The present invention is directed to a composition of a phenylalkylamino carbamate compound comprising an admixture of the compound with an effective amount of one or more excipients wherein at least one excipient is dibasic calcium phosphate dihydrate, whereby the dibasic calcium phosphate dihydrate reduces degradation of the phenylalkylamino carbamate compound in the composition.

Therefore, in one general aspect, the present invention provides a composition comprising an effective amount of one or more excipients wherein at least one excipient is dibasic calcium phosphate dihydrate and a compound of formula (I):

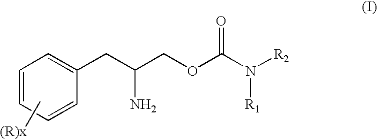

or a form thereof wherein
R is a member selected from the group consisting of hydrogen, alkyl of 1 to 8 carbon atoms, lower alkyl of 1 to 4 carbon atoms, halogen selected from F, Cl, Br and I, lower alkoxy containing 1 to 3 carbon atoms, nitro, hydroxy, trifluoromethyl and thioalkoxy containing 1 to 3 carbon atoms;
x is an integer selected from 1, 2 or 3, with the proviso that R may be the same or different when x is 2 or 3;
R₁ and R₂ can be the same or different from each other and are independently selected from the group consisting of hydrogen, alkyl of 1 to 8 carbon atoms, lower alkyl of 1 to 4 carbon atoms, aryl, arylalkyl and cycloalkyl of 3 to 7 carbon atoms;
alternatively, R₁ and R₂ can be joined to form a 5 to 7-membered heterocycle substituted with a member selected from the group consisting of hydrogen, alkyl and aryl, wherein the heterocycle can optionally comprise 1 to 2 additional nitrogen atom ring members and 0 to 1 oxygen atom ring members.

In an embodiment, the present invention provides a composition comprising an effective amount of one or more excipients wherein at least one excipient is dibasic calcium phosphate dihydrate and a carbamic acid 2-amino-3-phenyl-propyl ester compound of formula (Ia):

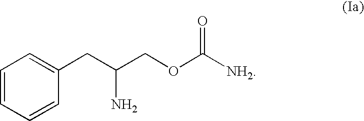

In another embodiment, the compositions of the present invention are tablets comprising an effective amount of dibasic calcium phosphate dihydrate and a carbamic acid 2-amino-3-phenyl-propyl ester compound of formula (Ia).

In another embodiment, the present invention provides a composition comprising an effective amount of one or more excipients wherein at least one excipient is dibasic calcium phosphate dihydrate and a carbamic acid (2R)-2-amino-3-phenyl-propyl ester compound of formula (Ib):

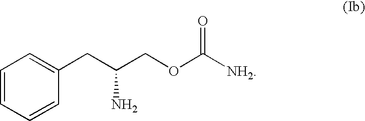

In another embodiment, the compositions of the present invention are tablets comprising an effective amount of dibasic calcium phosphate dihydrate and a carbamic acid (2R)-2-amino-3-phenyl-propyl ester compound of formula (Ib).

In another embodiment, carbamic acid (2R)-2-amino-3-phenyl-propyl ester compound of formula (Ib) predominates in a range of from about 75% or greater; or in a range of from about 90% or greater; or in a range of from about 95% or greater; or in a range of from about 98% or greater; or in a range of from about 99% or greater.

In another embodiment, the present invention provides a composition comprising an effective amount of one or more excipients wherein at least one excipient is dibasic calcium phosphate dihydrate and a carbamic acid (2S)-2-amino-3-phenyl-propyl ester compound of formula (Ic):

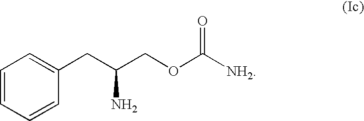

In another embodiment, the compositions of the present invention are tablets comprising an effective amount of dibasic calcium phosphate dihydrate and a carbamic acid (2S)-2-amino-3-phenyl-propyl ester compound of formula (Ic).

In another embodiment, carbamic acid (2S)-2-amino-3-phenyl-propyl ester compound of formula (Ic) predominates in a range of from about 75% or greater; or in a range of from about 90% or greater; or in a range of from about 95% or greater; or in a range of from about 98% or greater; or in a range of from about 99% or greater.

The present invention also provides methods of making and using the composition of the invention.

### DETAILED DESCRIPTION OF THE INVENTION

All publications cited herein are hereby incorporated by reference. Unless defined otherwise, all technical and scientific terms used herein have the same meaning as commonly understood to one of ordinary skill in the art to which this invention pertains.

The following abbreviations used in this specification have the following meanings: the term “API” means active pharmaceutical ingredient; “CNS” means central nervous system; “HPLC” means High Pressure Liquid Chromatography; and “RH” means Relative Humidity.

It must be noted that as used herein and in the appended claims, the singular forms “a,” “an,” and “the” include plural reference unless the context clearly dictates otherwise. Thus, for example, a reference to “a phenylalkylamino carbamate” is a reference to one or more phenylalkylamino carbamates and includes equivalents thereof known to those skilled in the art and so forth.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, the terms “comprising”, “containing”, “having” and “including” are used in their open, non-limiting sense.

As used herein, the term “composition” is intended to encompass a product comprising the specified ingredients in the specified amounts, as well as any product which results, directly or indirectly, from combinations of the specified ingredients in the specified amounts. Furthermore, the term composition is used interchangebly with the term “formulation,” whereby both terms are intended to have a similar meaning and both of which, in addition to the foregoing definition, are intended to take on the ordinary meaning given to them by one skilled in the art.

As used here in, the term “dibasic calcium phosphate dihydrate” or “DCPD” is a chemical compound having the formula of CaHPO₄.2H₂O. Synonyms and trademarks for dibasic calcium phosphate dihydrate include: Cafos; calcium hydrogen orthophosphate dihydrate; calcium monohydrogen phosphate dihydrate; Calstar; Calipharm; dicalcium orthophosphate; Difos; DI-TAB; E341; Emcompress® (brand of DCPD); phosphoric acid calcium salt (1:1) dihydrate; secondary calcium phosphate; calcium phosphate; and dicalcium phosphate (DCP). The latter two terms are commonly used generic terms in the pharmaceutical art.

DCPD refers to commercially available grades of DCPD that are typically used in wet-granulated or roller-compacted formulations or in dry blend, direct-compression formulations. The milled grade of DCPD typically has a pH of about 6.5 to a pH of about 7. The unmilled grade of DCPD typically has a pH of about 5.4.

DCPD is a white, odorless, tasteless, nonhygroscopic compound that is stable at room temperature. Under certain temperature and humidity conditions, DCPD loses water of crystallization below 100° C. Further, depending upon the degree of hydration, granulation (milled vs. unmilled) and the like, the surface pH of the DCPD changes.

In the present invention, the use of commercially available unmilled DCPD is contemplated, wherein the unmilled DCPD has a pH in a range of from about 5.0 to a pH of about 5.8; or a pH in a range of from about 5.1 to a pH of about 5.7; or a pH in a range of from about 5.2 to a pH of about 5.6; or a pH in a range of from about 5.3 to a pH of about 5.5; or a pH in a range of about 5.4.

In the present invention, the use of unmilled DCPD having a pH in one or more of the foregoing pH ranges has the function of significantly reducing degradation of a phenylalkylamino carbamate compound, thus resulting in improved stability of the compound. Such a function of unmilled DCPD is dependent on the structure of the compound and the presence of reactive groups.

DCPD can be used in both tablet and capsule formulations. DCPD may also be used both as an excipient and as a source of calcium in nutritional supplements. As a tablet excipient, DCPD is used because of its compaction properties and good-flow properties, particularly the unmilled material.

The term “tablet” means an API mixed with excipients and pressed into an oral dosage form.

A “capsule” is an oral dosage form in the shape of an oblong rounded container containing an API optionally mixed with excipients.

An “excipient” is generally an inactive substance used as a vehicle for an API. In addition, excipients can be used to aid the process by which a product is manufactured. An excipient is generally inactive, however, depending on the physical and chemical stability of the API, certain excipients can either degrade the API or can be used to stabilize the API. In a composition, using standard formulation techniques, the API may be dissolved or mixed with one or more optional excipients. The types of excipients used in a tablet include, but are not limited to, binders, fillers, disintegrants, lubricants, coatings, sweeteners, and flavors and colors. In many instances, one particular excipient may be used to perform more than one function, e.g., a binder may be used as a filler. In other instances, not every excipient is physically and chemically compatible with every API.

In addition, depending on the route of administration, taste of the drug or dosage form, various excipients may be used to enhance the pharmaceutical elegance of the composition.

A “binder” is generally an inactive ingredient used to hold the ingredients in a tablet together. A wide variety of binders can be used, including but not limited to, gum, wax, tapioca starch (cassava flour), polyethylene glycol, hydroxypropyl methylcellulose (HPMC), hydroxypropyl cellulose, and polyvinylpyrrolidone, etc. In some instances, a binder may be used as a filler.

A “filler” is generally an inactive substance used to fill out the size and shape of a tablet or capsule, making it practical to produce and convenient for the consumer to use, i.e., making a product bigger or easier to handle. Examples of fillers include, but are not limited to, cellulose, lactose, sucrose, mannitol, DCPD, microcrystalline cellulose (MCC), HPMC, soybean oil, safflower oil, ProSolv HD90 (brand of a co-processed mixture of MCC and colloidal silicon dioxide) and the like. In some instances, a binder may be used as a filler; for example, the binder cellulose or HPMC may be used as a filler in tablets or hard gelatin capsules. In another example, soybean or safflower oil is used as the filler in soft gelatin capsules.

A “disintegrant” is generally an inactive ingredient added to the tablet that readily absorbs water to help the tablet disperse once swallowed. A disintegrant expands when wet causing the tablet to break apart in the digestive tract, thus releasing the drug for absorption. Examples of disintegrants include, but are not limited to, sodium starch glycolate (SSG) and cross-linked polyplasdone (CLP or crospovidone). Some binders, such as starch, are also used as disintegrants.

A “lubricant” is generally an inactive ingredient added to prevent other ingredients from clumping together and from sticking to equipment. Examples of lubricants include, but are not limited to, common minerals, talc, silica, stearic acid (stearin), magnesium stearate (MS), sodium lauryl sulfate (SLS), sodium stearyl fumarate (SSF) and colloidal silicon dioxide (CSD) and the like.

A “powder flow enhancer” or “glidant” is generally an inactive ingredient that functions as the name implies. Examples of lubricants that function as powder flow enhancers are CSD and talc.

The term “form” means, in reference to a compound of the present invention, that such may exist as, without limitation, a salt, stereoisomer, tautomer, crystalline, polymorph, amorphous, solvate, hydrate, ester, prodrug or metabolite form. The present invention encompasses all such compound forms and mixtures thereof.

The term “isolated form” means, in reference to a compound of the present invention, that such may exist in an essentially pure state such as, without limitation, an enantiomer, a racemic mixture, a geometric isomer (such as a cis or trans stereoisomer), a mixture of geometric isomers and the like. The present invention encompasses all such compound forms and mixtures thereof.

The compounds of the invention may be present in the form of pharmaceutically acceptable salts or esters. For use in medicines, the term “pharmaceutically acceptable salts or esters” shall mean non-toxic salts or esters of the compounds employed in this invention which are generally prepared by reacting the free acid with a suitable organic or inorganic base. Examples of such salts include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, triethiodide, valerate and the like.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each isolated specie rotates the plane of polarized light in the opposite direction such that the mixture is devoid of optical activity.

The term “enantiomer” means an isomer having a nonsuperimposable mirror image. The term “diastereomer” means stereoisomers that are not enantiomers.

The term “chiral” means a molecule which, in a given configuration, cannot be superimposed on its mirror image. This is in contrast to achiral molecules which can be superimposed on their mirror images.

The two distinct mirror image versions of the chiral molecule are also known as levo (left-handed), abbreviated L, or dextro (right handed), abbreviated D, depending on which way they rotate polarized light. The symbols “R” and “S” represent the atom configuration of groups around a stereogenic carbon atom(s) and are intended to be used as defined in the literature.

An isolated form of a chiral mixture means those forms that are substantially free of one mirror image molecule. Such substantially pure forms include those wherein one mirror image is present in a range of less than 25% in the mixture, of less than 10%, of less than 5%, of less than 2% or less than 1%.

An example of an enantiomerically enriched form isolated from a racemic mixture includes a dextrorotatory enantiomer, wherein the mixture is substantially free of the levorotatory isomer. In this context, substantially free means the levorotatory isomer may, in a range, comprise less than 25% of the mixture, less than 10%, less than 5%, less than 2% or less than 1% of the mixture according to the formula:

$$\% levorotatory = \frac{( mass levorotatory )}{( mass dextrorotatory ) + ( mass levorotatory )} \times 100$$

Similarly, an example of an enantiomerically enriched form isolated from a racemic mixture includes a levorotatory enantiomer, wherein the mixture is substantially free of the dextrorotatory isomer. In this context, substantially free means the dextrorotatory isomer may, in a range, comprise less than 25% of the mixture, less than 10%, less than 5%, less than 2% or less than 1% of the mixture according to the formula:

$$\% dextrorotatory = \frac{( mass dextrorotatory )}{( mass dextrorotatory ) + ( mass levorotatory )} \times 100$$

The compounds of the invention may be prepared as individual isomers by either isomer-specific synthesis or resolved from an isomeric mixture.

Furthermore, compounds of the present invention may have at least one crystalline, polymorph or amorphous form. The plurality of such forms are intended to be included in the scope of the invention. In addition, some of the compounds may form solvates with water (i.e., hydrates) or common organic solvents (e.g., organic esters such as ethanolate and the like). The plurality of such solvates are also intended to be encompassed within the scope of this invention.

The term “alkyl” means a saturated aliphatic branched or straight-chain hydrocarbon radical or linking group having from 1 up to 8 carbon atoms in a linear or branched arrangement. The term “alkyl” also includes a “lower alkyl” radical or linking group having from 1 up to 4 carbon atoms respectively, such as methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Alkyl radicals may be attached to a core molecule and further substituted on any atom when allowed by available valences.

The term “alkoxy” means an alkyl radical or linking group having from 1 up to 8 carbon atoms in a linear or branched arrangement, wherein the radical or linking group is attached through an oxygen linking atom, as in the formula: —O-alkyl. The term “alkoxy” also includes a “lower alkoxy” radical or linking group having from 1 up to 4 carbon atoms respectively, such as methoxy, ethoxy, propoxy, butoxy and the like. An alkoxy radical may be attached to a core molecule and further substituted on any carbon atom when allowed by available valences.

The term “thioalkoxy” means an alkoxy or lower alkoxy radical or linking group, wherein the radical or linking group is attached through a sulfur linking atom, as in the formula: —S-alkyl. A thioalkoxy radical may be attached to a core molecule and further substituted on any carbon atom when allowed by available valences.

The term “cycloalkyl” means a saturated or partially unsaturated cyclic hydrocarbon ring system radical, wherein the ring system may have from 3 to 12 carbon atom ring members. The term “cycloalkyl” also includes ring systems having from 3 to 7 ring members, 3 to 10 ring members, 5 to 6 ring members, 5 to 12 ring members, 9 to 12 ring members and the like, such as cyclopropyl, cyclobutyl, cyclopentyl, cyclohexyl, cycloheptyl, cyclooctyl, 1H-indenyl, indanyl, 9H-fluorenyl, 1,2,3,4-tetrahydro-naphthalenyl, acenaphthenyl, adamantanyl and the like. Cycloalkyl radicals may be attached to a core molecule and further substituted on any atom when allowed by available valences.

The term “aryl” means an unsaturated aromatic hydrocarbon ring system radical. Aryl ring systems include phenyl, naphthalenyl, azulenyl, anthracenyl and the like. Examples of aryl in compounds representative of the present invention include phenyl or naphthalenyl. Aryl radicals may be attached to a core molecule and further substituted on any atom when allowed by available valences.

The term “arylalkyl” means an aryl ring system radical attached through an alkyl linking group, as in the formula: -alkyl-aryl.

The term “hetero”, when used as a prefix for a ring system, refers to the replacement of at least one carbon atom member in the ring system with a heteroatom selected from N, O, S, S(O), or SO₂. A hetero ring may have 1, 2, 3 or 4 carbon atom members replaced by a nitrogen atom. Alternatively, a ring may have 1, 2 or 3 nitrogen atom members and 1 oxygen or sulfur atom member. Alternatively, a ring may have 1 oxygen or sulfur atom member. Alternatively, up to two adjacent ring members may be heteroatoms, wherein one heteroatom is nitrogen and the other heteroatom is selected from N, S or O.

The term “heterocycle” means a saturated or partially unsaturated “hetero” ring system radical. Heterocyclyl ring systems include azetidinyl, 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azepanyl, hexahydro-1,4-diazepinyl, hexahydro-1,4-oxazepanyl, tetrahydro-furanyl, tetrahydro-thienyl, tetrahydro-pyranyl, tetrahydro-pyridazinyl, indolinyl (also referred to as 2,3-dihydro-indolyl), benzo[1,3]dioxolyl, 2,3-dihydro-1,4-benzodioxinyl, 2,3-dihydro-benzofuranyl, 1,2-dihydro-phthalazinyl and the like. Heterocycle radicals may be attached to a core molecule and further substituted on any atom when allowed by available valences.

A “tablet coating” protects tablet ingredients or tablet integrity from deterioration by moisture in the air and, in many cases, makes tablets easier to swallow. Some coatings are used to provide color or a smooth finish, or to facilitate printing on the tablet (although characters and symbols are easy to emboss into the tablets using special punches).

In one embodiment, a cellulose film coating is used which is free of sugar and potential allergy-causing substances. In another embodiment, other coating materials are used such as corn protein (zein) or an extraction from trees (pharmaceutical glaze).

Some tablets have a special coating termed an enteric coating, which is resistant to stomach acid and dissolves in the high pH of the intestines. The purpose of this coating is to prevent dissolution of the tablet in the stomach, where the stomach acid may degrade the active ingredient, or where the time of passage may compromise its effectiveness, in favor of dissolution in the small intestine, where the active principle is better absorbed.

A “release coating” controls the rate of drug release, or controls specifically when the drug will be released in the digestive tract. Coating is also used for product identification and differentiation.

As used herein, “ambient conditions” are the conditions measured in the immediate area surrounding a composition of the invention. This term can be applied to any unit of measure, such as temperature, pressure, humidity, light intensity, etc. For example, ambient conditions can be used to refer to a combination of a given temperature and relative humidity, such as 25° C. and 20% RH.

Under certain conditions of elevated temperature and relative humidity, such as, 25° C. and 40% RH, 25° C. and 60% RH, 25° C. and 80% RH, 45° C. and 20% RH, 45° C. and 40% RH, 45° C. and 60% RH, 45° C. and 80% RH, or 40° C. and 75% RH and the like, an exposed compound or composition may be subject to degradation.

In this invention, it has been discovered that unmilled DCPD provides protection against degradation of a compound of formula (I), which is more susceptible to hydrolysis and rearrangement as pH is increased (as depicted in Scheme A, B and C).

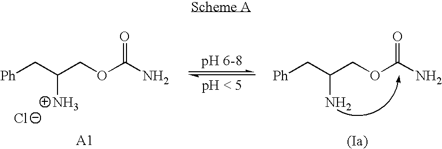

For a 1-carbamoyloxymethyl-2-phenyl-ethyl-ammonium chloride salt of Compound A1, a higher formulation pH shifts the equilibrium to provide the product carbamic acid 2-amino-3-phenyl-propyl ester of formula (Ia). As shown, the labile, free amine is subject to electrophilic cyclization.

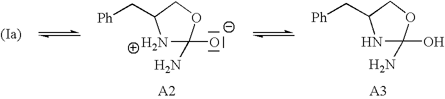

The compound of formula (Ia) is also in equilibirum with an intermediate Compound A2, which is likewise in equilibrium with an intermediate degradation product 2-amino-4-benzyl-oxazolidin-2-ol Compound A3.

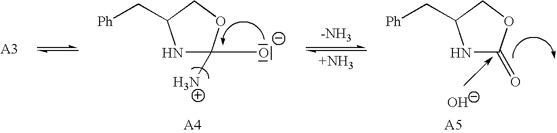

Compound A3 is further in equilibirum with an intermediate Compound A4. The removal of ammonia shifts the equilibrium to provide a first major degradation product 4-benzyl-oxazolidin-2-one Compound A5.

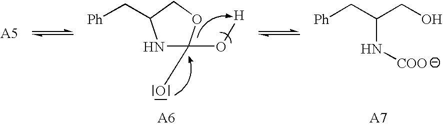

In a humid environment, the presence of free hydroxy ions available from water molecules shift the equilibrium of Compound A5 toward an intermediate Compound A6. The presence of free hydrogen ions also available from water shift the equilibrium of Compound A6, resulting in the ring opening, to provide a free (1-hydroxymethyl-2-phenyl-ethyl)-carbamic acid Compound A7.

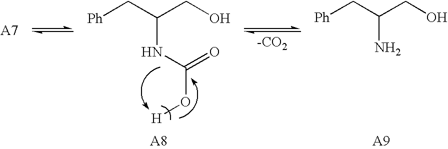

As degradation continues, free hydrogen ions further shift the equilibrium of Compound A7 toward an intermediate (1-hydroxymethyl-2-phenyl-ethyl)-carbamic acid Compound A8. The removal of carbon dioxide almost irreversibly shifts the equilibrium to provide a second major degradation product 2-amino-3-phenyl-propan-1-ol Compound A9.

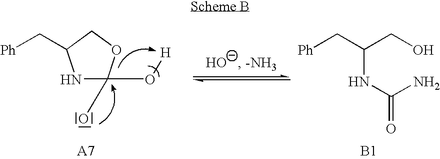

Free hydroxy ions and the removal of ammonia continue to shift the equilibrium of Compound A7 to provide a minor degradation product (1-hydroxymethyl-2-phenyl-ethyl)-urea Compound B1.

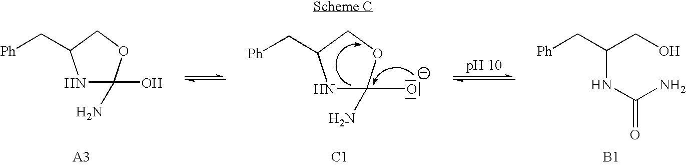

Compound A3 is also in equilibrium with an intermediate Compound C1. An increase in basic pH shifts the equilibrium to provide the minor degradation product Compound B1.

It will be appreciated that there will be potential improvements in shelf-life of compounds of formula (I) in a composition containing unmilled DCPD. Therefore, in one general aspect, the present invention provides a composition comprising an effective amount of unmilled dibasic calcium phosphate dihydrate and a compound of formula (I).

As used herein, an “effective amount of dibasic calcium phosphate dihydrate” means that amount of DCPD added to a composition that makes a compound of formula (I) stable in the composition. For example, an “effective amount of dibasic calcium phosphate dihydrate” can be the amount of DCPD added to a composition that decreases the physical or chemical degradation of a compound of formula (I) in the composition. It is readily appreciated that the effective amount of DCPD can vary depending upon the particular compound of formula (I), the dose range of the compound and the presence of other excipients in the composition, etc. Methods are known in the art for determining the “effective amount of DCPD”. For example, a skilled artisan can determine the effective amount of DCPD experimentally by making blends containing a compound of formula (I), DCPD and other excipients, subjecting the blends to elevated temperature and relative humidity storage for accelerated degradation, and measuring the amount of compound degradation.

The “effective amount of DCPD” is about 4% (w/w) of the composition to obtain the benefit of the invention. Furthermore, embodiments intended to be included within the scope of the present include an “effective amount of DCPD” of about 4% (w/w), 6% (w/w), 8% (w/w), 10% (w/w), 12% (w/w), 14% (w/w), 16% (w/w), 18% (w/w), 20% (w/w), 22% (w/w), 24% (w/w), 26% (w/w), 28% (w/w), 30% (w/w), 32% (w/w), 34% (w/w), 36% (w/w), 38% (w/w), 40% (w/w), 42% (w/w), 44% (w/w), 46% (w/w), 48% (w/w), 50% (w/w), 60% (w/w), 70% (w/w), and the like of the composition.

Embodiments of the present invention include an effective amount of DCPD in a range of from about 4% (w/w) to about 40% (w/w), a range of from about 4% (w/w) to about 35% (w/w), a range of from about 4% (w/w) to about 30% (w/w), a range of from about 4% (w/w) to about 25% (w/w) , a range of from about 4% (w/w) to about 20% (w/w), a range of from about 4% (w/w) to about 10% (w/w) and a range of about 4%.

The term “stable” as used herein, refers to the tendency of a compound or a composition to remain substantially in the same physical and chemical form for a period of 6 months; or, a period of one year; or, a period of two years; or, a period of 3 years; or, a period of 4 years; or, a period of 5 years, when stored under ambient conditions.

Embodiments of the present invention include compositions that remain stable for a period of time in a range of about 6 months to about 5 years; or, in a range of from about one year to about 5 years; or, in a range of from about 2 years to about 5 years; or, in a range of from about 3 years to about 5 years; or, in a range of from about 4 years to about 5 years; or, in a range of about 5 years, when stored under ambient conditions.

In another embodiment, the present invention provides a tablet comprising a compound of formula (I) and an effective amount of DCPD. The invention is not limited by the tabletting method. The tablets of the present invention can be formed by either the wet-granulated method or by a dry blend, direct-compression tabletting method.

In still another embodiment, the present invention provides a tablet comprising a compound of formula (I) and an effective amount of commercially available unmilled DCPD prepared in a dry granulation and a direct compression tabletting method.

The composition of the present invention can optionally further comprise additional diluents or excipients and other therapeutic agents.

Embodiments of the present invention include a composition further comprising an additional excipient selected from MCC, HPMC, mannitol, SSG, CLP, SLS, SSF or CSD.

For example, a composition of the present invention can comprise a carbamic acid (2R)-2-amino-3-phenyl-propyl ester compound of formula (Ib) as the API, MCC or HPMC as a binder or filler, DCPD as a filler and SSG or CLP as the disintegrant. The tablet can further optionally comprise one or more of talc, SLS, SSF or CSD for use as a wetting agent or powder flow enhancer.

Another embodiment of the present invention includes a composition comprising one or more of an excipient selected from HPMC and CLP.

In another embodiment, the composition of the present invention comprises other therapeutic agents. Such compositions are especially of interest in the treatment of CNS disorders. Therefore, embodiments of the invention include a composition comprising an effective amount of dibasic calcium phosphate dihydrate, a compound of formula (I), and a therapeutic agent selected from the group consisting of: selective serotonin reuptake inhibitors (SSRI's), selective serotonin and norepinephrine reuptake inhibitors (SNRI's), older tricyclic antidepressants (TCAs), monoamine oxidase inhibitors (MAO-inhibitors), reversible inhibitors of monoamine oxidase (RIMAs), tertiary amine tricyclics and secondary amine tricyclic antidepressants.

Embodiments of the invention also include a composition comprising an effective amount of dibasic calcium phosphate dihydrate, a compound of formula (I), and a therapeutic agent selected from the group consisting of: fluoxetine, duloxetine, venlafaxine, milnacipran, citalopram, fluvoxamine, paroxetine, sertraline, 5-MCA-NAT, lithium carbonate (LiCO₃), isocarboxazid, pheneizine, tranylcypromine, selegiline, moclobemide, opioid receptor antagonists, selective neurokinin antagonists, corticotropin releasing factor (CRF) antagonists, antagonists of tachykinins, α-adrenoreceptor antagonists, amitriptyline, clomipramine, doxepin, imipramine, venlafaxine, trimipramine, amoxapine, desipramine, maprotiline, nortriptyline and protriptyline and pharmaceutically acceptable salts thereof.

The present invention also provides a method of preparing the composition of the invention comprising the step of admixing an effective amount of one or more excipients wherein at least one excipient is DCPD with a compound of formula (I). The compositions may be conveniently presented in unit dosage forms, and prepared by any methods known in the art of pharmacy.

To prepare the pharmaceutical compositions of this invention, one or more compounds of formula (I) or salt thereof as the active ingredient is intimately admixed with an effective amount of DCPD and a pharmaceutically acceptable carrier according to conventional pharmaceutical compounding techniques. Carriers are generally necessary and inert pharmaceutical excipients, including, but not limited to, binders, fillers, disintegrants, suspending agents, lubricants, flavorings, sweeteners, preservatives, dyes and coatings. In preparing compositions in oral dosage form, any of the usual pharmaceutical carriers may be employed which provide a stable dosage form. For example, for solid oral preparations, suitable carriers and additives include starches, sugars, diluents, granulating agents, lubricants, binders, disintegrating agents and the like.

Any solid form of a compound of formula (I) can be used in the invention including, but not limited to, a salt, stereoisomer (such as an enantiomer or a racemic mixture), tautomer, crystalline, polymorph, amorphous, solvate, hydrate, ester, prodrug or metabolite form. The present invention encompasses all such compound forms and mixtures thereof.

Commercially available grades of unmilled DCPD are commonly used in direct compression/compaction or dry granulation techniques and are used in the present invention.

The compounds of formula (I) can be synthesized by methods known to those skilled in the art, as described in U.S. Pat. Nos. 5,705,640, 5,756,817, 5,955,499 and 6,140,532, which are hereby incorporated by reference in their entirety.

The salts and esters of the compounds of Formula (I) can be produced by treating the compound with an acid in suitable solvent or by means well known to those of skill in the art.

The invention also provides the use of a composition of the invention, for example, in the treatment of CNS disorders. The term “CNS disorders” means a disorder selected from CNS disorders, such as pain, depression, anxiety, epilepsy, stroke, dementia and Parkinson's disease.

The invention further provides the use of an effective amount of DCPD and a compound of formula (I) in the manufacture of a medicament for the treatment of CNS disorders.

The present invention further provides a method for the treatment of CNS disorders in a subject in need thereof comprising administering to the subject a therapeutically or prophylactically effective amount of a composition comprising an effective amount of dibasic calcium phosphate dihydrate and a compound of formula (I). The method also comprises administering to the subject a prophylactically effective amount of a composition comprising an effective amount of dibasic calcium phosphate dihydrate and a compound of formula (I).

The terms “subject” and “patient” are used herein interchangeably and as used herein refer to an animal, preferably a mammal, and most preferably a human, who has been the object of treatment, observation or experiment. The term mammals include human patients and non-human primates, as well as experimental animals such as rabbits, rats, mice and other like animals.

Therefore, the term “a subject in need of treatment” as used herein will refer to a subject or patient who currently has or may develop a CNS disorder, including any mood disorder which can be treated by a therapeutic agent, or any other disorder in which the patient's present clinical condition or prognosis could benefit from the administration of one or more compounds of Formula (I) alone or in combination with another therapeutic intervention including but not limited to another therapeutic agent.

The term “therapeutically effective amount” as used herein means a sufficient amount of one or more of the compounds of the invention to produce a therapeutic effect, as defined above, in a subject or patient in need of such treatment.

The term “prophylactically effective amount” is intended to mean that amount of a pharmaceutical drug that will prevent or reduce the risk of occurrence of the biological or medical event that is sought to be prevented in a tissue or a system, animal or human that is being sought by a researcher, veterinarian, medical doctor or other clinician.

Methods are known in the art for determining therapeutically and prophylactically effective doses for the instant pharmaceutical composition. For example, for use as an adjunct for treating CNS disorders, the compound can be employed at a daily dose in the range of about 0.1 mg to 400 mg usually in a regimen of 1 to 2 times per day, for an average adult human. The effective amount, however, may be varied depending upon the particular compound used, the mode of administration, the strength of the preparation and the advancement of the disease condition. In addition, factors associated with the particular patient being treated, including patient age, weight, diet, time of administration and response to treatment, will result in the need to adjust dosages.

Because of their ease in administration, tablets and capsules represent the most advantageous oral dosage unit form for the composition of the present invention. If desired, tablets may be sugar coated or enteric coated by standard techniques. The tablets or capsules can be coated or otherwise compounded to provide a dosage form affording the advantage of prolonged action. For example, the tablet or pills can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The composition of the present invention may be used in a unit dosage form such as a tablet, capsule, powder or granule.

The pharmaceutical compositions herein will contain, per dosage unit, e.g., tablet, capsule or powder, an amount of the active ingredient necessary to deliver a therapeutically or prophylactically effective dose as described above. For example, the pharmaceutical compositions herein can contain, per unit dosage unit, a therapeutically or prophylactically effective dose in a range of from about 25 to about 400 mg of the active ingredient, or a dose in a range of from about 50 to about 200 mg of the active ingredient.

In some embodiments of the present invention, compositions of this invention may be administered as a combination product either singly or concomitantly with one or more other compound or therapeutic agent, e.g., with other antidepressant agents. In these embodiments, the present invention provides methods to treat or prevent CNS disorders in a patient. The method includes the step of; administering to the patient in need of treatment a therapeutically or prophylactically effective amount of one of the compounds of formula (I) disclosed herein in combination with an effective amount of one or more other compounds or therapeutic agents that have the ability to augment or synergistically augment the therapeutic effects of the compounds of the present invention.

“Concomitant administration” or “combination administration” of a compound, therapeutic agent or known drug with a composition of the present invention means administration of one or more other therapeutic agents and, in addition, the one or more compositions of the invention at such time that both the other therapeutic agents and the compound of formula (I) will have a therapeutic effect. In some cases this therapeutic effect will be synergistic. Such concomitant administration can involve concurrent (i.e. at the same time), prior, or subsequent administration of the therapeutic agent with respect to the administration of a compound of the present invention. A person of ordinary skill in the art would have no difficulty determining the appropriate timing, sequence and dosages of administration for particular therapeutic agents and compounds of the present invention.

In addition, in some embodiments, the composition of the present invention may be used, either alone or in combination with one or more other therapeutic agents as described above, or their salts or esters, for manufacturing a medicament for the purpose of providing adjuvant treatment to a patient or subject in need thereof.

This invention will be better understood by reference to the examples that follow. Those skilled in the art will readily appreciate that these examples are only illustrative of the invention as described more fully in the claims that follow thereafter.

### EXAMPLE 1

### Excipient Compatibility Study

Determination of possible incompatibilities between an API and different excipients is an important aspect of development of a solid oral dosage form. In order to develop a robust composition, an excipient compatibility study is designed and performed.

The general design of an excipient compatibility study involves an experiment where a systematic selection of all possible combinations of excipients selected for a particular API are tested. Each composition blend comprises the excipients selected but omits one excipient until all combinations of selected excipients have been tested according to the formula:

$${\sum }_{j = 1}^{k} {\prod }_{i {= 1}_{i \neq j}}^{k} {l}_{i}$$

where k defines the number of excipient classes and each excipient class has a level lⱼ, where the level j is the series: 1,2, . . . , k. In this case, the sum k is 4, where the selection of excipients corresponds to filler, disintegrant, lubricant and flow enhancer.

The typical composition of a tablet formulation consists of the API and excipients, such as a binder, a filler, a disintegrant and a powder flow enhancer or a lubricant. For this experiment, four fillers (DCPD, MCC, mannitol and lactose), two disintegrants (CLP and SSG), two lubricants (magnesium stearate and SSF) and a powder flow enhancer (CSD) were mixed with the carbamic acid (2R)-2-amino-3-phenyl-propyl ester compound of formula (Ib). It is appreciated that experimental methods used herein are readily applicable to compositions comprising different APIs and different excipients.

The fillers were chosen on the basis of their flowability and compactability: two are water-soluble (lactose and mannitol) and two are water-insoluble (MCC and DCPD). In general, lactose is a desirable filler based on cost, flowability and purity. In this experiment, lactose was selected as a positive control because lactose is not physically or chemically compatible with the compound of formula (Ib), since lactose is a reducing sugar and the compound of formula (Ib) has a labile amino group. The aldehyde reactive tautomer of lactose very likely reacts with the amino group of the compound of formula (Ib) and results in physical and chemical degradation of the compound and composition thereof.

All excipients tested were obtained from commercial sources: DCPD (JRS Pharma, Patterson, N.Y.); lactose (Foremost, Rothschild, Wis.); mannitol (SPI Polyols, Newark, Del.); MCC (FMC Bioploymer, Philadelphia, Pa.); CLP (ISP Technologies, Kalvert City, Ky.); sodium starch glycolate (JRS Pharma, Patterson, N.Y.); magnesium stearate (Mallinckrodt, St. Louis, Mo.); sodium stearyl fumarate (JRS Pharma, Patterson, N.Y.); colloidal silicon dioxide (Cabot, Tuscola, Ill.); Prosolv HD90 (JRS Pharma, Patterson, N.Y.) and talc (Whittaker, Clark and Daniels, S. Plainfield, N.J.).

The excipient compatibility study consisted of 36 composition blends. The API by itself was used as a control (Blend No. 37). The API and excipients, in the same proportion as they would appear in a tablet dosage form, were weighed and delumped, if necessary, using a #20 mesh screen. The ingredients were sequentially added into a mortar according to the order: API, filler, disintegrant, lubricant and powder flow enhancer. The blend samples were filled into 1 ounce amber glass bottles. All bottles containing the blends remained open and were covered individually with a single layer of thin paper towel for to allow equilibration of humidity inside the bottle.

74 bottles were placed at 60° C. and 75% RH, 210 bottles at 40° C. and 75% RH, 74 bottles at 25° C. and 60% RH, and 37 bottles at 4° C. At predetermined time points, samples were pulled out of the specific chambers, allowed to equilibrate at room temperature for 2 hrs and analyzed. The samples at 60° C. and 75% RH were removed at 15 and 30 days, and 40° C. and 75% RH were removed at 1, 2, 3, and 6 months for the analyses of physical appearance, impurities, degradants, enantiomeric purity and weight loss/gain. The samples at 25° C. and 75% RH were kept in a passive state and never tested. The samples at 4° C. were used as controls for appearance testing.

For physical appearance analyses, a small portion of the blend was removed from the bottle and arranged on an 8×5 grid. All 37 blends were compared at the same time.

For HPLC analyses, a small portion of the blend (approximately 200 mg containing 50 mg of the compound of formula (Ib)) was removed from the bottle, weighed accurately and placed in a 200 mL volumetric flask. 125 mL of sample solvent (80:20 v/v 0.1% o-phosphoric acid: methanol) was added to each flask and the flasks were vigorously shaken for 30 minutes. Following shaking, the solution was brought up to the mark by adding additional amounts of sample solvent. The flasks were stoppered and inverted 20 times for ensuring complete mixing of the blend. A 5 mL aliquot was removed from the flask by a syringe. Following removal of the solution from the flask, a 0.45 micron filter was placed on the syringe tip. After discarding the first 3 mL of the liquid through the tip, 1 mL was collected in a glass HPLC vial. Each vial was immediately closed and all the samples were subsequently assayed by HPLC.

The HPLC setup consisted of a Waters Xterra MS C₁₈ column, 4.6×100 mm column dimensions, 3.5 μm particle size; Column Temperature: 35° C.; Flow Rate: 1.0 mL/min; Detection: UV 215 nm; Run Time: 45 min; Injection Volume: 10 μL; Mobile Phase: Preparation and composition; Mobile Phase A: 0.1% H₃PO₄; Mobile Phase B: Acetonitrile; Retention Time: Approximately 4 to 7 min.

The statistical analysis of the study results was carried out through a series of non-independent ANOVAs, each ANOVA corresponding to a subset of runs with each subset characterized by the removal of 1 excipient class. For example, if the level lⱼ is k, then there were k excipient classes. In this case, there were four excipient classes, resulting in four ANOVAs carried out. The error term was estimated from the residual error. Graphical methods were used to enable scientific interpretation of the results.

From the physical appearance analyses, it was observed that blends containing DCPD and mannitol (without any fillers) had a reduced degree of degradation, as shown by lack of discoloration (appeared white) when stored at 40° C. and 75% RH for 3 months (Table 1B & 1C respectively).

Blends using MCC as the filler appeared slightly discolored (light brown, Table 1A).

Depending on the ingredients of the other excipients, blends containing lactose as the filler appeared from light brown to dark brown (Table 1D).

The following codes are used in the tables:
SSF sodium stearyl fumarate
MS magnesium stearate
CLP cross-linked polyplasdone (crospovidone)
SSG sodium starch glycolate
CSD colloidal silicon dioxide
W white
VLB very light brown
LB light brown
B brown
DB dark brown
ND not detected
LOD limit of detection
API carbamic acid (2R)-2-amino-3-phenyl-propyl ester
A5 4-benzyl-oxazolidin-2-one (degradation product Compound A5)
A9 2-amino-3-phenyl-propan-1-ol (degradation product Compound A9)
App appearance

All blends were stored at 40° C. and 75% RH for 3 months and analyzed at the start of the study (Initial) and at the one month and three month timepoints.

TABLE 1A
Blends containing MCC

| Blend | Added | | API assay | A5 assay | A9 assay | |
|---|---|---|---|---|---|---|
| No. | Excipients | Time | (%) | (%) | (%) | App |
| 2 | CLP and SSF | Initial | 99.51 | ND | ND | W |
| | | 1 Mo | 99.20 | 0.14 | 0.08 | LB |
| | | 3 Mo | 98.54 | 0.39 | 0.10 | LB |
| 3 | CLP and MS | Initial | 100.13 | ND | ND | W |
| | | 1 Mo | 97.13 | 1.83 | 0.09 | LB |
| | | 3 Mo | 98.76 | 0.18 | 0.09 | LB |
| 4 | SSG and SSF | Initial | 99.67 | ND | ND | W |
| | | 1 Mo | 95.22 | 0.18 | 0.09 | LB |
| | | 3 Mo | 95.42 | 0.50 | 0.12 | LB |
| 5 | SSG and MS | Initial | 99.75 | ND | ND | W |
| | | 1 Mo | 96.40 | 0.14 | 0.09 | LB |
| | | 3 Mo | 97.09 | 0.42 | 0.10 | LB |
| 18 | CLP and CSD | Initial | 100.40 | ND | ND | W |
| | | 1 Mo | 100.89 | LOD | 0.09 | LB |
| | | 3 Mo | 98.25 | 0.18 | 0.09 | LB |
| 19 | SSG and CSD | Initial | 100.12 | ND | ND | W |
| | | 1 Mo | 96.94 | 0.17 | 0.09 | LB |
| | | 3 Mo | 96.54 | 0.40 | 0.18 | LB |
| 26 | SSF and CSD | Initial | 97.78 | ND | ND | W |
| | | 1 Mo | 100.00 | 0.19 | 0.10 | LB |
| | | 3 Mo | 97.93 | 0.48 | 0.16 | LB |
| 27 | MS and CSD | Initial | 100.15 | ND | ND | W |
| | | 1 Mo | 100.8 | LOD | 0.09 | LB |
| | | 3 Mo | 98.25 | 0.2 | 0.17 | LB |

TABLE 1B
Blends containing DCPD

| Blend | Added | | API assay | A5 assay | A9 assay | |
|---|---|---|---|---|---|---|
| No. | Excipients | Time | (%) | (%) | (%) | App |
| 6 | CLP and SSF | Initial | 97.46 | ND | ND | W |
| | | 1 Mo | 99.41 | ND | 0.08 | W |
| | | 3 Mo | 103.21 | 0.18 | 0.13 | W |
| 7 | CLP and MS | Initial | 98.27 | ND | ND | W |
| | | 1 Mo | 96.23 | ND | 0.09 | W |
| | | 3 Mo | 96.47 | ND | 0.08 | W |
| 8 | SSG and SSF | Initial | 99.03 | ND | ND | W |
| | | 1 Mo | 92.54 | ND | 0.09 | W |
| | | 3 Mo | 99.95 | 0.20 | 0.07 | W |
| 9 | SSG and MS | Initial | 98.24 | ND | ND | W |
| | | 1 Mo | 98.25 | ND | 0.09 | W |
| | | 3 Mo | 97.40 | 0.17 | 0.07 | W |
| 20 | CLP and CSD | Initial | 99.99 | ND | ND | W |
| | | 1 Mo | 97.85 | ND | 0.08 | W |
| | | 3 Mo | 98.10 | 0.15 | 0.16 | W |
| 21 | SSG and CSD | Initial | 100.02 | ND | ND | W |
| | | 1 Mo | 93.33 | ND | 0.07 | W |
| | | 3 Mo | 94.29 | 0.19 | 0.12 | W |
| 28 | SSF and CSD | Initial | 98.87 | ND | ND | W |
| | | 1 Mo | 99.13 | ND | 0.09 | W |
| | | 3 Mo | 96.08 | 0.19 | 0.14 | W |
| 29 | MS and CSD | Initial | 99.63 | ND | ND | W |
| | | 1 Mo | 99.24 | ND | 0.09 | W |
| | | 3 Mo | 99.69 | ND | 0.15 | W |

TABLE 1C
Blends containing Mannitol

| Blend | Added | | API assay | A5 assay | A9 assay | |
|---|---|---|---|---|---|---|
| No. | Excipients | Time | (%) | (%) | (%) | App |
| 10 | CLP and SSF | Initial | 100.74 | ND | ND | W |
| | | 1 Mo | 95.79 | 0.13 | 0.09 | W |
| | | 3 Mo | 96.07 | 0.29 | 0.07 | W |
| 11 | CLP and MS | Initial | 97.54 | ND | ND | W |
| | | 1 Mo | 96.98 | 0.16 | 0.09 | W |
| | | 3 Mo | 99.01 | 0.24 | 0.09 | W |
| 12 | SSG and SSF | Initial | 99.55 | ND | ND | W |
| | | 1 Mo | 96.10 | 0.27 | 0.09 | W |
| | | 3 Mo | 98.23 | 0.67 | 0.12 | W |
| 13 | SSG and MS | Initial | 99.84 | ND | ND | W |
| | | 1 Mo | 102.13 | 0.18 | 0.09 | W |
| | | 3 Mo | 93.60 | 0.66 | 0.13 | W |
| 22 | CLP and CSD | Initial | 94.93 | ND | ND | W |
| | | 1 Mo | 101.41 | ND | 0.09 | W |
| | | 3 Mo | 98.46 | ND | 0.15 | W |
| 23 | SSG and CSD | Initial | 98.88 | ND | ND | W |
| | | 1 Mo | 94.69 | 0.18 | 0.08 | W |
| | | 3 Mo | 94.87 | 0.50 | 0.17 | W |
| 30 | SSF and CSD | Initial | 98.87 | ND | ND | W |
| | | 1 Mo | 95.63 | 0.17 | 0.09 | W |
| | | 3 Mo | 98.46 | 0.31 | 0.17 | W |
| 31 | MS and CSD | Initial | 99.62 | ND | ND | W |
| | | 1 Mo | 95.63 | 0.17 | 0.09 | W |
| | | 3 Mo | 96.93 | 0.27 | 0.16 | W |

TABLE 1D
Blends containing Lactose

| Blend | Added | | API assay | A5 assay | A9 assay | |
|---|---|---|---|---|---|---|
| No. | Excipients | Time | (%) | (%) | (%) | App |
| 14 | CLP and SSF | Initial | 99.19 | ND | ND | W |
| | | 1 Mo | 98.74 | ND | 0.09 | ND |
| | | 3 Mo | 96.44 | 0.13 | 0.05 | B |
| 15 | CLP and MS | Initial | 98.29 | ND | ND | W |
| | | 1 Mo | 98.43 | ND | 0.08 | ND |
| | | 3 Mo | 97.34 | ND | 0.07 | B |
| 16 | SSG and SSF | Initial | 99.14 | ND | ND | W |
| | | 1 Mo | 96.87 | ND | 0.09 | ND |
| | | 3 Mo | 91.63 | 0.26 | 0.07 | DB |
| 17 | SSG and MS | Initial | 99.92 | ND | ND | W |
| | | 1 Mo | 94.24 | ND | 0.08 | ND |
| | | 3 Mo | 89.14 | 0.26 | 0.06 | DB |
| 24 | CLP and CSD | Initial | 99.61 | ND | ND | W |
| | | 1 Mo | 98.71 | ND | ND | ND |
| | | 3 Mo | 98.11 | ND | 0.14 | LB |
| 25 | SSG and CSD | Initial | 99.42 | ND | ND | W |
| | | 1 Mo | 94.46 | ND | 0.09 | ND |
| | | 3 Mo | 89.13 | 0.26 | 0.14 | DB |
| 32 | SSF and CSD | Initial | 98.53 | ND | ND | W |
| | | 1 Mo | 95.22 | ND | 0.09 | ND |
| | | 3 Mo | 99.46 | ND | 0.14 | B |
| 33 | MS and CSD | Initial | 100.25 | ND | ND | W |
| | | 1 Mo | 95.45 | ND | 0.09 | ND |
| | | 3 Mo | 100.22 | ND | 0.14 | B |

When the blends were analyzed by HPLC for chemical degradation, the blends containing DCPD (Table 1B) were found chemically to be more stable than blends containing MCC (Table 1A), mannitol (Table 1C) or lactose (Table 1D).

Two degradation products of the carbamic acid (2R)-2-amino-3-phenyl-propyl ester compound of formula (Ib) were found by HPLC: 4-benzyl-oxazolidin-2-one Compound A5 and 2-amino-3-phenyl-propan-1-ol Compound A9.

Two of the fillers showed substantial color change at 3 months. These color changes were reflected by corresponding losses in assay potency.

The effects of different lubricants or disintegrants had a visually significant effect when lactose was the filler. Using a Least Squares Means analysis to estimate the loss in potency over 3 months indicated that the filler lactose, combined with disintegrant sodium starch glycolate (SSG) was by far the least stable formulation, losing 9.5% potency over 3 months. This combination of filler and disintegrant also produced the greatest color change to dark brown among all formulations.

Stability was improved when the disintegrant cross-linked polyplasdone (CLP or crospovidone) was used in place of SSG. For those formulations, the potency loss was reduced to 1.7%, however the color still changed to brown.

The filler microcrystalline cellulose (MCC), in combination with either disintegrant SSG or CLP, also showed a color change to light brown at 3 months. The chemical potency loss when using MCC as the filler ranged from 1 to 4% at 3 months.

Use of the fillers, DCPD and mannitol, showed no color change at 3 months. Both of these fillers in combination with CLP reported changes in potency of less than 1% on average over 3 months, compared with use of SSG where the potency loss was approximately 2 to 4%. Mannitol afforded less protection compared with DCPD, affording the least loss in potency in combination with either disintegrant. The combinations of DCPD and CLP as a disintegrant reported the least loss in potency.

### EXAMPLE 2

### Tablet Formulation Study

Based on the results of the excipient compatibility study described in Example 1, mannitol and DCPD were determined to be fillers that were compatible with the other excipients tested. To further compare DCPD and mannitol, four tablet formulations were prepared by employing strategies that were likely to be used in commercial manufacturing of tablets.

Formulation 119, 120 and 121 were prepared using direct compression. in these blends, HPMC was added as a dry binder and a coarse grade of MCC was used. Talc was added as a fluidizing agent during fluid bed granulation. Prosolv HD90 was used as the filler.

Formulation 120 contained DCPD as the filler. The other three formulations (formulation nos. 119, 121 and 131) contained mannitol as the filler.

Formulation 131 was prepared as a wet granulation blend. The disintegrant was added after granulation.

The samples were maintained at 40° C. and 75% RH for 40 days in closed and opened bottles. Appearance was visually inspected at various timepoints and the results are shown in Table 2. For the results of each appearance inspection, the first letter represents the closed bottles and the second letter represents the opened bottles.

TABLE 2
Tablet Formulations and Appearance Results

| Ingredient | 119 | | 120 | | 121 | | 131 | |
|---|---|---|---|---|---|---|---|---|
| DCPD | No | | 202.0 mg | | No | | No | |
| Mannitol | 202.0 mg | | No | | 202.0 mg | | 168.0 mg | |
| MCC | 150.0 mg | | 150.0 mg | | No | | No | |
| HPMC | 18.0 mg | | 18.0 mg | | 18.0 mg | | 12.0 mg | |
| CLP | 18.0 mg | | 18.0 mg | | 18.0 mg | | 12.0 mg | |
| MS | 6.0 mg | | 6.0 mg | | 6.0 mg | | 6.0 mg | |
| CSD | 6.0 mg | | 6.0 mg | | No | | No | |
| HD90 | No | | No | | 156.0 mg | | No | |
| Talc | No | | No | | No | | 4.0 mg | |
| App Day 0 | W | W | W | W | W | W | W | W |
| App Day 2 | VLB | W | W | W | W | W | VLB | W |
| App Day 6 | LB | VLB | VLB | W | LB | LB | VLB | VLB |
| App Day 9 | B | VLB | VLB | VLB | LB | LB | VLB | VLB |
| App Day 20 | B | B | LB | LB | B | B | B | B |
| App Day 40 | DB | B | B | B | B | DB | B | DB |

From the physical appearance and HPLC analyses, it was observed that formulation 120 showed less physical and chemical degradation, being visually less discolored than the other formulations, at the 1 month timepoint.

It is to be understood that the preceding description of the invention and various examples thereof have emphasized certain aspects. Numerous other equivalents not specifically elaborated on or discussed may nevertheless fall within the spirit and scope of the present invention or the following claims and are intended to be included.

What is claimed is:

1. A composition of a phenylalkylamino carbamate compound comprising an admixture of the compound with an effective amount of one or more excipients wherein at least one excipient is dibasic calcium phosphate dihydrate, whereby the dibasic calcium phosphate dihydrate reduces degradation of the phenylalkylamino carbamate compound in the composition.

2. The composition of claim 1, wherein the compound is a compound of formula (I): 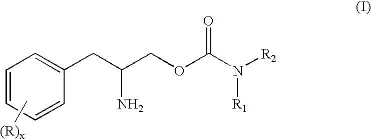
or a form thereof wherein
R is a member selected from the group consisting of hydrogen, alkyl of 1 to 8 carbon atoms, lower alkyl of 1 to 4 carbon atoms, halogen selected from F, Cl, Br and I, lower alkoxy containing 1 to 3 carbon atoms, nitro, hydroxy, trifluoromethyl and thioalkoxy containing 1 to 3 carbon atoms;
x is an integer selected from 1, 2 or 3, with the proviso that R may be the same or different when x is 2 or 3;
R₁ and R₂ can be the same or different from each other and are independently selected from the group consisting of hydrogen, alkyl of 1 to 8 carbon atoms, lower alkyl of 1 to 4 carbon atoms, aryl, arylalkyl and cycloalkyl of 3 to 7 carbon atoms;
alternatively, R₁ and R₂ can be joined to form a 5 to 7-membered heterocycle substituted with a member selected from the group consisting of hydrogen, alkyl and aryl, wherein the heterocycle can optionally comprise 1 to 2 additional nitrogen atom ring members and 0 to 1 oxygen atom ring members.

3. The composition of claim 2, wherein said compound is carbamic acid 2-amino-3-phenyl-propyl ester.

4. The composition of claim 2, wherein said compound is carbamic acid (2R)-2-amino-3-phenyl-propyl ester.

5. The composition of claim 4, wherein said compound predominates in a range of from about 75% or greater; or in a range of from about 90% or greater; or in a range of from about 95% or greater; or in a range of from about 98% or greater; or in a range of from about 99% or greater.

6. The composition of claim 2, wherein said compound is carbamic acid (2S)-2-amino-3-phenyl-propyl ester.

7. The composition of claim 6, wherein said compound predominates in a range of from about 75% or greater; or in a range of from about 90% or greater; or in a range of from about 95% or greater; or in a range of from about 98% or greater; or in a range of from about 99% or greater.

8. The composition of claim 1, wherein said dibasic calcium phosphate dihydrate is unmilled.

9. The composition of claim 8, wherein said dibasic calcium phosphate dihydrate has a pH in a range of from about 5.0 to a pH of about 5.8; or a pH in a range of from about 5.1 to a pH of about 5.7; or a pH in a range of from about 5.2 to a pH of about 5.6; or a pH in a range of from about 5.3 to a pH of about 5.5; or a pH in a range of about 5.4.

10. The composition of claim 1, wherein said effective amount of dibasic calcium phosphate dihydrate is in a range of from about 4% (w/w) to about 40% (w/w).

11. The composition of claim 1, wherein said effective amount of dibasic calcium phosphate dihydrate is in a range of from about 4% (w/w) to about 35% (w/w).

12. The composition of claim 1, wherein said effective amount of dibasic calcium phosphate dihydrate is in a range of from about 4% (w/w) to about 30% (w/w).

13. The composition of claim 1, wherein said effective amount of dibasic calcium phosphate dihydrate is in a range of from about 4% (w/w) to about 25% (w/w).

14. The composition of claim 1, wherein said effective amount of dibasic calcium phosphate dihydrate is in a range of from about 4% (w/w) to about 20% (w/w).

15. The composition of claim 1, wherein said effective amount of dibasic calcium phosphate dihydrate is in a range of from about 4% (w/w) to about 10% (w/w).

16. The composition of claim 1, wherein said effective amount of dibasic calcium phosphate dihydrate is about 4% (w/w).

17. The composition of claim 1, wherein the composition remains stable for a period of time in a range of about 6 months to about 5 years; or, in a range of from about one year to about 5 years; or, in a range of from about 2 years to about 5 years; or, in a range of from about 3 years to about 5 years; or, in a range of from about 4 years to about 5 years; or, in a range of about 5 years, when stored under ambient conditions.

18. The composition of claim 1, wherein the excipients are selected from microcrystalline cellulose, hydroxypropyl methylcellulose, mannitol, sodium starch glycolate, cross-linked polyplasdone, sodium lauryl sulfate, sodium stearyl fumarate or colloidal silicon dioxide.

19. The composition of claim 18, wherein the excipients are selected from microcrystalline cellulose, hydroxypropyl methylcellulose, sodium starch glycolate or cross-linked polyplasdone.

20. The composition of claim 1, wherein the excipients are selected from hydroxypropyl methylcellulose or cross-linked polyplasdone.

21. The composition of claim 1, wherein said composition is a tablet.

22. The composition of claim 21, wherein the excipients are selected from microcrystalline cellulose, hydroxypropyl methylcellulose, mannitol, sodium starch glycolate, cross-linked polyplasdone, sodium lauryl sulfate, sodium stearyl fumarate or colloidal silicon dioxide.

23. The composition of claim 21, wherein the excipients are selected from microcrystalline cellulose, hydroxypropyl methylcellulose, sodium starch glycolate or cross-linked polyplasdone.

24. The composition of claim 21, wherein the excipients are selected from hydroxypropyl methylcellulose or cross-linked polyplasdone.

25. The composition of claim 21, wherein the compound is the compound of claim 3.

26. The composition of claim 21, wherein the compound is the compound of claim 4.

27. The composition of claim 26, wherein said compound predominates in a range of from about 75% or greater; or in a range of from about 90% or greater; or in a range of from about 95% or greater; or in a range of from about 98% or greater; or in a range of from about 99% or greater.

28. The composition of claim 1, further comprising one or more therapeutic agents.

29. The composition of claim 28, wherein the therapeutic agents are selected from selective serotonin reuptake inhibitors, selective serotonin and norepinephrine reuptake inhibitors, tricyclic antidepressants, monoamine oxidase inhibitors, reversible inhibitors of monoamine oxidase, tertiary amine tricyclics and secondary amine tricyclic antidepressants.

30. The composition of claim 28, wherein the therapeutic agents are selected from fluoxetine, duloxetine, venlafaxine, milnacipran, citalopram, fluvoxamine, paroxetine, sertraline, 5-MCA-NAT, lithium carbonate, isocarboxazid, phenelzine, tranylcypromine, selegiline, moclobemide, opioid receptor antagonists, selective neurokinin antagonists, corticotropin releasing factor antagonists, antagonists of tachykinins, α-adrenoreceptor antagonists, amitriptyline, clomipramine, doxepin, imipramine, venlafaxine, trimipramine, amoxapine, desipramine, maprotiline, nortriptyline and protriptyline and pharmaceutically acceptable salts thereof.

31. A method of preparing a composition comprising the step of admixing an effective amount of one or more excipients wherein at least one excipient is dibasic calcium phosphate dihydrate with a compound of formula (I): 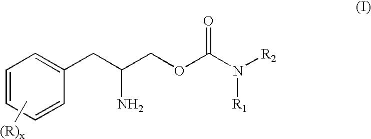
or a form thereof wherein
R is a member selected from the group consisting of hydrogen, alkyl of 1 to 8 carbon atoms, lower alkyl of 1 to 4 carbon atoms, halogen selected from F, Cl, Br and I, lower alkoxy containing 1 to 3 carbon atoms, nitro, hydroxy, trifluoromethyl and thioalkoxy containing 1 to 3 carbon atoms;
x is an integer selected from 1, 2 or 3, with the proviso that R may be the same or different when x is 2 or 3;
R₁ and R₂ can be the same or different from each other and are independently selected from the group consisting of hydrogen, alkyl of 1 to 8 carbon atoms, lower alkyl of 1 to 4 carbon atoms, aryl, arylalkyl and cycloalkyl of 3 to 7 carbon atoms;
alternatively, R₁ and R₂ can be joined to form a 5 to 7-membered heterocycle substituted with a member selected from the group consisting of hydrogen, alkyl and aryl, wherein the heterocycle can optionally comprise 1 to 2 additional nitrogen atom ring members and 0 to 1 oxygen atom ring members.

32. The method of claim 31, wherein the compound is the compound of claim 3.

33. The method of claim 31, wherein the compound is the compound of claim 4.

34. The method of claim 33, wherein said compound predominates in a range of from about 75% or greater; or in a range of from about 90% or greater; or in a range of from about 95% or greater; or in a range of from about 98% or greater; or in a range of from about 99% or greater.

35. A method for treatment of a CNS disorder in a subject in need thereof comprising the step of administering to the subject a therapeutically or prophylactically effective amount of a composition comprising an effective amount of one or more excipients wherein at least one excipient is dibasic calcium phosphate dihydrate and a compound of formula (I): 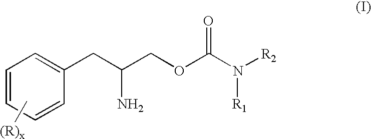
or a form thereof wherein
R is a member selected from the group consisting of hydrogen, alkyl of 1 to 8 carbon atoms, lower alkyl of 1 to 4 carbon atoms, halogen selected from F, Cl, Br and I, lower alkoxy containing 1 to 3 carbon atoms, nitro, hydroxy, trifluoromethyl and thioalkoxy containing 1 to 3 carbon atoms;
x is an integer selected from 1, 2 or 3, with the proviso that R may be the same or different when x is 2 or 3;
R₁ and R₂ can be the same or different from each other and are independently selected from the group consisting of hydrogen, alkyl of 1 to 8 carbon atoms, lower alkyl of 1 to 4 carbon atoms, aryl, arylalkyl and cycloalkyl of 3 to 7 carbon atoms;
alternatively, R₁ and R₂ can be joined to form a 5 to 7-membered heterocycle substituted with a member selected from the group consisting of hydrogen, alkyl and aryl, wherein the heterocycle can optionally comprise 1 to 2 additional nitrogen atom ring members and 0 to 1 oxygen atom ring members.

36. The method of claim 35, wherein the compound is the compound of claim 3.

37. The method of claim 35, wherein the compound is the compound of claim 4.

38. The method of claim 37, wherein said compound predominates in a range of from about 75% or greater; or in a range of from about 90% or greater; or in a range of from about 95% or greater; or in a range of from about 98% or greater; or in a range of from about 99% or greater.

39. The method of claim 35, wherein the CNS disorder is selected from pain, depression, anxiety, epilepsy, stroke, dementia and Parkinson's disease.

40. A composition resulting from a method of preparation comprising the step of admixing an effective amount of one or more excipients wherein at least one excipient is dibasic calcium phosphate dihydrate with a compound of formula (I): 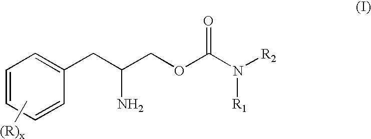
or a form thereof wherein
R is a member selected from the group consisting of hydrogen, alkyl of 1 to 8 carbon atoms, lower alkyl of 1 to 4 carbon atoms, halogen selected from F, Cl, Br and I, lower alkoxy containing 1 to 3 carbon atoms, nitro, hydroxy, trifluoromethyl and thioalkoxy containing 1 to 3 carbon atoms;
x is an integer selected from 1, 2 or 3, with the proviso that R may be the same or different when x is 2 or 3;
R₁ and R₂ can be the same or different from each other and are independently selected from the group consisting of hydrogen, alkyl of 1 to 8 carbon atoms, lower alkyl of 1 to 4 carbon atoms, aryl, arylalkyl and cycloalkyl of 3 to 7 carbon atoms;
alternatively, R₁ and R₂ can be joined to form a 5 to 7-membered heterocycle substituted with a member selected from the group consisting of hydrogen, alkyl and aryl, wherein the heterocycle can optionally comprise 1 to 2 additional nitrogen atom ring members and 0 to 1 oxygen atom ring members.

41. The composition of claim 40, wherein the compound is the compound of claim 3.

42. The composition of claim 40, wherein the compound is the compound of claim 4.

43. The composition of claim 42, wherein said compound predominates in a range of from about 75% or greater; or in a range of from about 90% or greater; or in a range of from about 95% or greater; or in a range of from about 98% or greater; or in a range of from about 99% or greater.

44. A tablet comprising an effective amount of dibasic calcium phosphate dihydrate and one or more excipients selected from microcrystalline cellulose, hydroxypropyl methylcellulose, mannitol, sodium starch glycolate, cross-linked polyplasdone, sodium lauryl sulfate, sodium stearyl fumarate or colloidal silicon dioxide and a compound of formula (I): 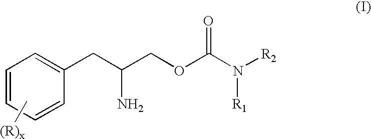
or a form thereof wherein
R is a member selected from the group consisting of hydrogen, alkyl of 1 to 8 carbon atoms, lower alkyl of 1 to 4 carbon atoms, halogen selected from F, Cl, Br and I, lower alkoxy containing 1 to 3 carbon atoms, nitro, hydroxy, trifluoromethyl, and thioalkoxy containing 1 to 3 carbon atoms;
x is an integer selected from 1, 2 or 3, with the proviso that R may be the same or different when x is 2 or 3;
R₁ and R₂ can be the same or different from each other and are independently selected from the group consisting of hydrogen, alkyl of 1 to 8 carbon atoms, lower alkyl of 1 to 4 carbon atoms, aryl, arylalkyl, cycloalkyl of 3 to 7 carbon atoms;
alternatively, R₁ and R₂ can be joined to form a 5 to 7-membered heterocycle substituted with a member selected from the group consisting of hydrogen, alkyl and aryl, wherein the heterocycle can optionally comprise 1 to 2 additional nitrogen atom ring members and 0 to 1 oxygen atom ring members.

45. The tablet of claim 44, wherein the compound is the compound of claim 3.

46. The tablet of claim 44, wherein the compound is the compound of claim 4.

47. The tablet of claim 46, wherein said compound predominates in a range of from about 75% or greater; or in a range of from about 90% or greater; or in a range of from about 95% or greater; or in a range of from about 98% or greater; or in a range of from about 99% or greater.

48. The tablet of claim 44, wherein the excipients are selected from microcrystalline cellulose, hydroxypropyl methylcellulose, sodium starch glycolate or cross-linked polyplasdone.

49. The tablet of claim 44, wherein the excipients are selected from hydroxypropyl methylcellulose or cross-linked polyplasdone.

50. The tablet of claim 44, wherein said effective amount of dibasic calcium phosphate dihydrate is in a range of from about 4% (w/w) to about 40% (w/w).

51. The tablet of claim 44, wherein said effective amount of dibasic calcium phosphate dihydrate is in a range of from about 4% (w/w) to about 35% (w/w).

52. The tablet of claim 44, wherein said effective amount of dibasic calcium phosphate dihydrate is in a range of from about 4% (w/w) to about 30% (w/w).

53. The tablet of claim 44, wherein said effective amount of dibasic calcium phosphate dihydrate is in a range of from about 4% (w/w) to about 25% (w/w).

54. The tablet of claim 44, wherein said effective amount of dibasic calcium phosphate dihydrate is in a range of from about 4% (w/w) to about 20% (w/w).

55. The tablet of claim 44, wherein said effective amount of dibasic calcium phosphate dihydrate is in a range of from about 4% (w/w) to about 10% (w/w).

56. The tablet of claim 44, wherein said effective amount of dibasic calcium phosphate dihydrate is about 4% (w/w).

57. Use of the composition of claim 1 in the manufacture of a medicament for the treatment of CNS disorders.

58. The use of claim 57, wherein the CNS disorder is selected from convulsions, epilepsy, stroke and muscle spasm; useful in the treatment of central nervous system diseases, particularly as anticonvulsants, antiepileptics, neuroprotective agents and centrally acting muscle relaxants; useful in treating and preventing neuropathic pain, cluster and migraine headache pain, bipolar disorder, chronic and acute neurodegenerative disorders, psychotic disorders, movement disorders, addictive disorders, impulse control disorders, anxiety disorders, antiepileptogenesis and for the treatment of pain.
